(19) United States
(12) Patent Application Publication
(10) Pub. No.: US 2015/0099003 A1
(43) Pub. Date: Apr. 9, 2015

(54) Topiramate Sustained-Release Pharmaceutical Composition, Method for Preparing Same, and Uses Thereof

(71) Applicant: Institute of Pharmacology and Toxicology Academy of Military Medical Sciences P.L.A. China, Beijing (CN)

(72) Inventors: Song Li, Beijing (CN); Chunsheng Gao, Beijing (CN); Wu Zhong, Beijing (CN); Yuli Wang, Beijing (CN); Meiyan Yang, Beijing (CN); Li Shan, Beijing (CN); Xinbo Zhou, Beijing (CN); Zhibing Zheng, Beijing (CN); Xiaokui Wang, Beijing (CN)

(73) Assignee: Institute of Pharmacology and Toxicology Academy of Military Medical Sciences P.L.A. China, Beijing (CN)

(21) Appl. No.: 14/386,173

(22) PCT Filed: Mar. 7, 2013
(86) PCT No.: PCT/CN2013/072283

§ 371 Date: Sep. 18, 2014

(30) Foreign Application Priority Data
Mar. 23, 2012 (CN) 201210080716.8

(51) Int. Cl. A61K 31/357; A61K 9/50

(52) U.S. Cl. CPC A61K 31/357; A61K 9/5078; A61K 9/5042; A61K 9/5026; A61K 9/5089
USPC 424/495; 549/387; 424/490; 514/454; 427/2.16

(57) ABSTRACT

A sustained-release pharmaceutical composition of topiramate, which is free of binding agent. The sustained-release pharmaceutical composition of topiramate is a sustained-release pellet, comprising a blank pellet core, a drug layer, and a sustained-release coating layer.

### TECHNICAL FIELD

The present invention pertains to medicine and chemical fields, and relates to sustained-release pharmaceutical composition of topiramate, its preparation method and uses. Specifically, the sustained-release pharmaceutical composition of topiramate is sustained-release pellet.

### BACKGROUND ART

Topiramate (2,3,4,5-bis-O-(1-methylethylene)-β-D-fructopyranose sulfamate) (as shown in the following Formula 1) is a broad spectrum nerve therapeutic agent approved by FDA in 1995, and has been used in clinic for many years for treatment of some epileptic seizures and prevention of migraine headache (E. Faught, et al., (1996) Neurology 46:1684-1690), and many documents disclosed the good therapeutic effects of topiramate in treatment of diabetes (U.S. Pat. No. 7,109,174B2 and U.S. Pat. No. 6,362,220B1), dysneuria (U.S. Pat. No. 6,908,902B2), depression (U.S. Pat. No. 6,627,653B2), mental disorders (U.S. Pat. No. 6,620,819B2), headache (U.S. Pat. No. 6,319,903B1) and hypertension (U.S. Pat. No. 6,201,010B1).

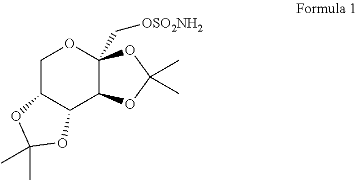

Topiramate is white crystalline powder, has bitter taste, is freely soluble in organic solvents such as acetone, chloroform, dimethylsulfoxide and ethanol, very freely soluble in alkaline solution having pH of 9-10 such as sodium hydroxide or sodium phosphate solutions, very slightly soluble in water (room temperature) with solubility of only about 9.8 mg/mL, and its saturated solution has pH value of 6.3 (Physician's Desk Reference, 56.sup.th ed., pp. 2590-2595 (2002)).

Topiramate has linear pharmacokinetic features, can be rapidly and completely absorbed in vivo. After being orally taken in dose of 400 mg by health volunteer, it can reach average plasma peak concentration (Cmax) within 2 h. Topiramate shows linear relation between blood concentration and dose in daily dose range of 100 mg to 800 mg, and has a low clearance for oral administration (22-36 ml/min) and a long plasma half-life (19-25 h). In plasma concentration range of 0.5-250 μm/ml, topiramate has a human plasma protein binding ratio of 15-41%, which decreases with the increase of plasma concentration.

At present, the dosage forms of topiramate used in clinic are normal tablets and capsules, in which the tablets have 4 specifications, i.e., 25 mg, 50 mg, 100 mg, and 200 mg; the capsules are sprinkle capsules and have 2 specifications, i.e., 15 mg and 25 mg, and since they have to be orally administrated for several times and dosages should be regulated, their administration is complicated and patients have poor compliance. More important, topiramate has a narrow therapeutic window, because the fluctuation of blood concentration usually results in some adverse reactions, which are mainly symptoms associated with central nervous system, such as ataxia, attention impairment, confusion, dizziness, fatigue, paresthesia, somnolence, and thinking abnormal, etc. (Physician's Desk Reference, 60th ed., pp 2538-2447(2006)).

Thus, in order to elevate compliance in patients and improve pharmaceutical efficiency, it is necessary to provide a sustained-release dosage form of topiramate that can reduce fluctuation of blood concentration and needs only one drug administration per day.

Oral sustained-release preparations, especially oral sustained-release pellets, have technical feature of “dose distribution”, so that the drug is distributed more homogeneously in gastrointestinal tract, and absorbed more uniformly. In addition, the reduction of administration number renders drug-time curve relatively smooth, reduces occurrence rate of toxic and side effects, and significantly improve compliance in patients, so that they are very important in pharmaceutical market and very popular in doctors and patients.

CN1988889A discloses a sustained-release preparation prepared by secondary granules, in which solid dispersion granules of topiramate are firstly prepared by melting method, then sustained-release granules are prepared by using sustained-release materials and the solid dispersion granules via one-step granulation or wet granulation methods, which has high production cost and complicated process.

CN102112126A discloses a sustained-release composition of low-dose topiramate which is used in combination with low-dose rapid-release phentermine for treatment of obesity, the sustained-release composition is prepared by firstly preparing topiramate drug-loading matrix cores (i.e., topiramate drug-loading cores) with 40% w/w of topiramate and 56.5% w/w of microcrystalline cellulose (AvicelPH102) via extrusion-spheronization method using 3.5% w/w of methyl cellulose (Methocel A15LV, MC) as a binding agent, then coating the topiramate drug-loading cores with 5.47% w/w of ethyl cellulose as sustained-release coating film material and 2.39% w/w of Povidone K30 (PVP K30) as a pore-forming agent, and finally forming topiramate pellets with controlled-release function.

WO2008061226A2 discloses sustained-release pellets of topiramate, which is prepared by performing drug-loading on surface of inert pellets such as sugar pellets with an aqueous dispersion containing 10-20% (w/w) topiramate and 0.5-4% HPMC or other binding agents via a fluidized bed coating method, then performing controlled-release coating on surface of the topiramate-loaded pellets, and further discloses that when inert pellets have a small particle diameter or pellets with high drug-loading rate are desired, high concentration of binding agent is necessary.

However, as for the above prepared sustained-release or controlled-release pellets (or pellets) of topiramate, the processes for preparing topiramate drug-loading pellet cores (or called as topiramate drug-loading matrix cores) all use a binding agent, such as HPMC or MC, etc., which means the increase of possibility of compatibility reaction with main drug topiramate, and in the meantime, the binding agent may influence dissolution state of the main drug and result in fluctuation of drug-release rate and effects on controlled-release. In addition, the adhesion degree of pellets would increase and the yield would decrease when a binding agent is added to solution during the process for loading drug on blank pellets.

Hence, it is extremely needed to provide a sustained-release preparation of topiramate which has desired drug release, high stability, and high yield, and can be readily prepared.

### DESCRIPTION OF THE INVENTION

With deep studying and inventive work, the inventors obtained a novel topiramate sustained-release composition (e.g., sustained- and controlled-release pellets), the topiramate sustained-release composition is free of a binding agent, and the inventors surprisingly found that the sustained-release pharmaceutical composition of topiramate has good sustained-release effects, high controllability, high stability, good repeatability, simple prescription, easy in operation and manufacture, and thus the following invention is provided.

The present invention provides a topiramate sustained-release pharmaceutical composition, which is a sustained-release pellet. The composition comprises: a) a blank pellet core; b) a drug layer, the drug layer is free of a binding agent; c) a sustained-release coating layer, in which the active drug layer is located on surface of the blank pellet core, and the sustained-release coating layer covers the external surface of the active drug layer. The schematic diagram of the sustained-release is shown in FIG. 1.

In the composition of the present invention, the blank pellet cores refer to pellet cores without physiological activity, may include but not be limited to sugar pellets, microcrystalline pellets, starch pellets, or silicon dioxide pellets, etc., preferably sugar pellets. The blank pellet core has diameter of 150 μm-1500 μm, preferably 300 μm-1000 μm, more preferably 400 μm-850 μm, most preferably 610 μm-750 μm. The blank pellet cores can be commercially available in market, or prepared by conventional means such as extrusion spheronization method, fluidized bed method.

In the composition of the present invention, the active drug layer is free of a binding agent, in which the binding agent includes starch slurry, syrup, polyvinylpyrrolidone (povidone, PVP, such as PVP K30), methyl cellulose (MC), ethyl cellulose (EC), highly-substituted hydroxypropyl cellulose (HPC), hydroxypropyl methyl cellulose (HPMC), sodium carboxymethyl cellulose, gelatin, Arabic gum, etc. When a blank pellet core is loaded with drug without using a binding agent, the time for loading drug is short, and the adhesion degree of pellets is low.

In the composition of the present invention, the sustained-release coating layer comprises a sustained-release coating material, including but not being limited to ethyl cellulose, Eudragit NE 30D, Eudragit RS 30D, or Eudragit RL30D, or a mixture thereof, preferably ethyl cellulose and Eudragit NE 30D, most preferably ethyl cellulose. The coating layer can further comprises a plasticizer, a pore-forming agent, an anti-sticking agent, a coloring agent, a light-screening agent, a flavoring agent, a sweetening agent, etc., in which the plasticizer includes but is not limited to glycerol, propylene glycol, polyethylene glycol, glycerol triacetate, triethyl citrate, phthalates or dibutyl sebate or a mixture thereof, preferably glycerol triacetate; the pore-forming agent includes but is not limited to polyethylene glycols, povidone, sucrose, salts, hydroxypropyl methyl cellulose, hydroxypropyl methyl cellulose or a mixture thereof, preferably povidone (PVP K30); the anti-sticking agent includes but is not limited to talc powder, magnesium stearate, aerosil or a mixture thereof, preferably talc powder; the light-screening agent includes but is not limited to titanium dioxide, etc.; the coloring agent includes but is not limited to iron oxide yellow, iron oxide red, coccinellin, lemon yellow, sunset yellow, indigo blue, etc.; the flavoring agent includes but is not limited to mint essence, lemon essence, orange essence, eucalyptol, caryophyllene alcohol, etc.; and the sweetening agent includes but is not limited to aspartame, vanillin, sorbitol, mannitol, artificial essences, or a mixture thereof.

In the present invention, the sustained-release coating has a weight increment (weight percentage of sustained-release coating film material to total composition, w/w) range that can be determined by tests, and generally, the weight increment range of the sustained-release coating is 2%-30%, preferably 4%-15%, more preferably 5%-10%, relative to the total weight of the composition.

In the composition of the present invention, the active ingredient (topiramate) is in an amount of 10%-50%, preferably 15%-45%, more preferably 20%-40%, relative to the total weight of the composition.

Further, the applicants found after a plenty of experiments that the above topiramate-carried pellet using the sustained-release coating layer comprising ethyl cellulose and PVP K30 in combination brought about unexpected good effects, compared with that of other sustained-release coating layers. That is, the topiramate pellet prepared with ethyl cellulose and PVP K30 as sustained-release coating layer material has better stability in drug release, which ensures consistency of drug release in different batches of samples, and the expected sustained-release effect can be achieved without heat treatment after coating. Thus, the coating process is simplified, and effects of heat treatment on drug release after coating are eliminated. The usage amount ratio of ethyl cellulose to PVP K30 is 1:0.20-1:0.45, preferably 1:0.25-1:0.40, especially preferably 1:0.3-1:0.35.

In a preferable embodiment of the present invention, the drug layer contains topiramate, the sustained-release coating layer uses ethyl cellulose as sustained-release coating material, PVP K30 as pore forming agent, and the usage amount ratio of ethyl cellulose to PVP K30 is 1:0.20-1:0.45.

In another preferable embodiment of the present invention, the drug layer contains topiramate, the sustained-release coating layer uses ethyl cellulose as sustained-release coating material, PVP K30 as pore forming agent, and the usage amount ratio of ethyl cellulose to PVP K30 is 1:0.20-1:0.45, and the range of weight increment of sustained-release coating is 5%-15%.

In another preferable embodiment of the present invention, the blank pellet core is sugar pellet, the drug layer contains topiramate, the sustained-release coating layer uses ethyl cellulose as sustained-release coating material, PVP K30 as pore forming agent, the usage amount ratio of ethyl cellulose to PVP K30 is 1:0.20-1:0.45, and the range of weight increment of sustained-release coating is 5%-15%.

In further another preferable embodiment of the present invention, the blank pellet core is sugar pellet having a particle diameter of 610 μm-750 μm, the drug layer contains topiramate, the sustained-release coating layer uses ethyl cellulose as sustained-release coating material, PVP K30 as pore forming agent, the usage amount ratio of ethyl cellulose to PVP K30 is 1:0.20-1:0.45, and the range of weight increment of sustained-release coating is 5%-10%.

In further another preferable embodiment of the present invention, the blank pellet core is sugar pellet having a particle diameter of 710 μm-850 μm, the drug layer uses topiramate as active drug, the sustained-release coating layer uses ethyl cellulose as sustained-release coating material, PVP K30 as pore forming agent, the usage amount ratio of ethyl cellulose to PVP K30 is 1:0.20-1:0.45, and the range of weight increment of sustained-release coating is 5%-8%.

In an particular embodiment of the present invention, the blank pellet core is sugar pellet having a particle diameter of 610 μm-750 μm, the drug layer contains topiramate, the sustained-release coating layer uses ethyl cellulose as sustained-release coating material, PVP K30 as pore forming agent, the usage amount ratio of ethyl cellulose to PVP K30 is 1:0.25-1:0.35, and the range of weight increment of sustained-release coating is 6%-8%.

In the present invention and the above embodiments, the drug layer contains topiramate, and further contains other pharmaceutically acceptable adjuvants, such as surfactants, disintegrating agents, flavoring agents, sweetening agents, anti-sticking agents, light-screening agents, etc. The surfactants include anionic surfactants, cationic surfactant, zwitterionic surfactants, and non-ionic surfactants, including but not being limited to sodium dodecyl sulfate, sodium hexadecanol sulfate, sodium octadecanol sulfate, sodium dodecylbenzene sulfonate, sodium dioctyl sulfosuccinate, sodium dihexyl sulfosuccinate, lecithin, sorbitan fatty acid esters, polyoxyethylene sorbitan fatty acid esters, polyoxyethylene fatty acid esters, polyoxyethylene fatty alcohol ethers, polymer of ethylene oxide and propylene oxide, polyoxyethylene 40 monostearate, polyoxyethylene 50 stearate, oxirane triblock copolymer, epoxypropane triblock copolymer, sorbitan monopalmitate (Span-40), sorbitan monostearate (Span-60), glyceryl monostearate, polyoxyethylene stearate, or mixtures thereof; the disintegrating agents include but are not limited to microcrystalline cellulose, low-substituted hydroxypropyl cellulose sodium, crosslinked polyvinylpyrrolidone, sodium carboxymethyl starch, pre-gelatinized starch, alginic acid, starch, effervescing disintegrants, or mixtures thereof; the anti-sticking agents include but are not limited to talc powder, magnesium stearate, aerosil, preferably talc powder; the light-screening agents include but are not limited to titanium dioxide, etc.; the flavoring agents include but are not limited to mint essence, lemon essence, orange essence, eucalyptol, caryophyllene alcohol, etc.; the sweetening agents include but are not limited to aspartame, vanillin, sorbitol, mannitol, artificial essences, etc.

The sustained-release pellet of topiramate of the present invention can bring about good therapeutic effect by once administration per 24 h, the in vivo blood concentration of drug is stable, peak concentration decreases significantly, and good sustained-release effect is achieved. The sustained-release pellet of topiramate of the present invention has in vitro release rate of: not greater than 35% within 1 h, between 30% and 60% within 4 h, between 60% and 90% within 8 h, and not less than 90% within in 16 h; preferably, not greater than 25% within 1 h, between 35% and 55% within 4 h, between 60% and 85% within 8 h, and not less than 90% within in 16 h; most preferably, not greater than 25% within 1 h, between 35% and 55% within 4 h, between 65% and 85% within 8 h, and not less than 90% within in 16 h.

The preferable conditions for determining the release rate in the present invention are in accordance with the first method (for sustained-release preparation or controlled-release preparation) of Release Rate Measurement (Appendix X D) of Part II of Chinese Pharmacopoeia, 2010 Edition, using apparatus as stated in the second method (slurry method) of Dissolution Rate Measurement (Appendix X C) of Part II of Chinese Pharmacopoeia, 2010 Edition, in which samples are taken and analyzed at different specified time points by using water (500 ml) as releasing media, at 37° C. and rotation speed of 100 rpm.

On the other hand, the present invention further provides a method for preparing a sustained-release pharmaceutical composition of topiramate, the method comprising:

a) providing ingredients of drug layer to perform drug-loading and coating a blank pellet core;

b) subjecting the drug-loading pellet to sustained-release coating.

Preferably, the method for preparing a sustained-release pellet of topiramate in the present invention comprises the following steps:

a) providing topiramate and other adjuvants of drug layer, adding with a suitable amount of solvent for dissolution, and performing drug-loading and coating a blank pellet core to obtain a drug-loading pellet;

b) subjecting the drug-loading pellet to sustained-release coating.

More preferably, the method for preparing a sustained-release pellet of topiramate in the present invention comprises the following steps:

a) providing topiramate and other adjuvants of drug layer, adding with a suitable amount of solvent for dissolution, and performing drug-loading and coating a blank pellet core with the drug solution;

b) dissolving a sustained-release coating material and other adjuvants of sustained-release coating layer in a solvent, subjecting the drug-loading pellet to sustained-release coating.

Most preferably, the method for preparing a sustained-release pellet of topiramate in the present invention comprises the following steps:

a) providing topiramate and other adjuvants of drug layer, adding with a suitable amount of solvent, heating and dissolving under stirring, providing a blank pellet core and placing in a fluidized bed coating pan for one-step granulation, performing drug-loading and coating with the above drug solution under stirring;

b) dissolving a sustained-release coating material and other adjuvants of sustained-release coating layer in a solvent, heating and dissolving under stirring, mixing homogeneously, passing through a 100 mesh sieve, to obtain a sustained-release coating solution;

c) taking the drug-loading pellet, spraying the sustained-release coating solution on surface of the drug-loading pellet in a fluidized bed, to obtain a sustained-release pellet of topiramate.

The suitable solvent for the method of the present invention is water, ethanol, acetone, propylene glycol, chloroform or a mixture thereof, preferably a mixture of water and ethanol, for example, 50% ethanol water solution, 70% ethanol water solution, 95% ethanol water solution.

In the method for preparing sustained-release pellet of topiramate of the present invention, the active ingredient in drug layer is topiramate, the drug layer does not contain a binding agent, in which the binding agent refers to starch slurry, syrup, polyvinylpyrrolidone (povidone, PVP, such as PVP K30), methyl cellulose (MC), ethyl cellulose (EC), highly-substituted hydroxypropyl cellulose (HPC), hydroxypropyl methyl cellulose (HPMC), sodium carboxymethyl cellulose, gelatin, Arabic gum, etc. The drug layer can further comprises other pharmaceutically acceptable adjuvants, such as surfactants, disintegrating agents, flavoring agents, sweetening agents, anti-sticking agents, light-screening agents, etc. The surfactant include anionic surfactants, cationic surfactant, zwitterionic surfactants, and non-ionic surfactants, including but not being limited to sodium dodecyl sulfate, sodium hexadecanol sulfate, sodium octadecanol sulfate, sodium dodecylbenzene sulfonate, sodium dioctyl sulfosuccinate, sodium dihexyl sulfosuccinate, lecithin, sorbitan fatty acid esters, polyoxyethylene sorbitan fatty acid esters, polyoxyethylene fatty acid esters, polyoxyethylene fatty alcohol ethers, polymer of ethylene oxide and propylene oxide, polyoxyethylene 40 monostearate, polyoxyethylene 50 stearate, oxirane triblock copolymer, epoxypropane triblock copolymer, sorbitan monopalmitate (Span-40), sorbitan monostearate (Span-60), glyceryl monostearate, polyoxyethylene stearate, or mixtures thereof; the disintegrating agents include but are not limited to microcrystalline cellulose, low-substituted hydroxypropyl cellulose sodium, crosslinked polyvinylpyrrolidone, sodium carboxymethyl starch, pregelatinized starch, alginic acid, starch, effervescing disintegrants, or mixtures thereof; the anti-sticking agents include but are not limited to talc powder, magnesium stearate, aerosil, preferably talc powder; the light-screening agents include but are not limited to titanium dioxide, etc.; the flavoring agents include but are not limited to mint essence, lemon essence, orange essence, eucalyptol, caryophyllene alcohol, etc.; the sweetening agents include but are not limited to aspartame, vanillin, sorbitol, mannitol, artificial essences, etc.

In the method for preparing the composition of the present invention, the sustained-release coating material in the sustained-release coating layer includes but is not limited to ethyl cellulose, Eudragit NE 30D, Eudragit RS 30D, or Eudragit RL30D, preferably ethyl cellulose and Eudragit NE 30D, most preferably ethyl cellulose. The coating layer further comprises a plasticizer, a pore-forming agent, an anti-sticking agent, a coloring agent, a light-screening agent, a flavoring agent, a sweetening agent, etc., in which the plasticizer includes but is not limited to glycerol, propylene glycol, polyethylene glycol, glycerol triacetate, triethyl citrate, phthalates or dibutyl sebate, preferably glycerol triacetate; the pore-forming agent includes but is not limited to polyethylene glycols, povidone, sucrose, salts, hydroxypropyl methyl cellulose, hydroxypropyl methyl cellulose, etc., preferably povidone (PVP K30); the anti-sticking agent includes but is not limited to talc powder, magnesium stearate, aerosil, preferably talc powder; the light-screening agent includes but is not limited to titanium dioxide, etc.; the coloring agent includes but is not limited to iron oxide yellow, iron oxide red, coccinellin, lemon yellow, sunset yellow, indigo blue, etc.; the flavoring agent includes but is not limited to mint essence, lemon essence, orange essence, eucalyptol, caryophyllene alcohol, etc.; and the sweetening agent includes but is not limited to aspartame, vanillin, sorbitol, mannitol, artificial essences, etc.

Preferably, the sustained-release coating layer of the sustained-release pellet of topiramate of the present invention contains ethyl cellulose and PVP K30.

In a specific embodiment of the present invention, the method for preparing pellet of topiramate comprises:

a) Topiramate is provided as main drug, dissolved with ethanol solution to prepare a solution with concentration of 20% (w/v) for drug-loading and coating. A blank pellet core is provided and placed in a fluidized bed coating pan for one step granulation, and the above drug solution is used for drug-loading and coating under stirring to obtain a drug-loading pellet core.

b) Ethyl cellulose as sustained-release coating material is dissolved in an ethanol solution to achieve a concentration of 3-8% (w/v), preferably 5-7% (w/v), and added with a suitable amount of specific pore-forming agent, PVP K30, then heated and dissolved under stirring, stirred homogenously, after passing through 100 mesh sieve, it is atomized and sprayed on the drug-loading pellet core with active drug layer of topiramate in a fluidized bed bottom-spraying coating pan to perform sustained-release coating.

The process parameters for drug-loading and sustained-release coating in the fluidized bed can be regulated according to practical situations, and preferable process parameters are as follows:

Drug-loading and coating —inlet air temperature is 50-70° C. (to keep pan internal temperature at 40±2° C.); inlet air pressure is 0.3-0.5 bar; atomization pressure is 1.0-2.0 bar; solution spray rate is 5-15 g/min.

Sustained-release coating —inlet air temperature is 40-45° C. (to keep pan internal temperature at 30-35° C.); inlet air pressure is 0.3-0.5 bar; atomization pressure is 1.0-2.0 bar, solution spray rate is 3-12 g/min.

The sustained-release pellet of topiramate of the composition of the present invention has a particle diameter of 150 μm-1500 μm, preferably 300 μm-1000 μm, more preferably 400 μm-850 μm, most preferably 610 μm-750 μm. The sustained-release pellet of topiramate of the composition of the present invention can be further processed to from other preparations, for example, can be loaded in capsules to form capsule preparation, or can be added with other pharmaceutically acceptable adjuvants and tableted to form tablets. It can also be combined with other active ingredients to form compound preparations.

The unit preparation of composition of the present invention can have a topiramate content of 1 mg-500 mg, preferably 5 mg-300 mg, more preferably 10 mg-250 mg, most preferably 20 mg-100 mg, optimally 23 mg-92 mg.

In one embodiment of the present invention, the unit preparation contains 23 mg of topiramate, and in another embodiment, the unit preparation contains 46 mg of topiramate, and in further another embodiment, the unit preparation contains 92 mg of topiramate.

The present invention further relates to a use of the sustained-release pharmaceutical composition of topiramate according to any one of items of the present invention in manufacture of a medicament for prophylaxis and/or treatment and/or adjunctive treatment of migraine, epilepsy, diabetes, dysneuria, depression, psychosis, headache, or hypertension.

The present invention further relates to a method for prophylaxis and/or treatment and/or adjunctive treatment of migraine, epilepsy, diabetes, dysneuria, depression, psychosis, headache, or hypertension, comprising a step of administering an effective amount of the sustained-release pharmaceutical composition of topiramate according to any one of items of the present invention.

In the present invention the subject to be administered is a subject, such as a mammal, including but not being limited to: human, monkey, pig, cattle, goat, etc.

In the present invention, the term “effective amount” refers to a dose that can fulfill treatment, prophylaxis, alleviation and/or remission of the diseases or disorders of the present invention in the subject.

### BRIEF DESCRIPTION OF THE DRAWINGS

FIG. 1: shows a schematic diagram of structure of topiramate pellet of the present invention.

FIG. 2: shows release rate curves of the 3 batches of topiramate sustained-release coating pellets of Example 6 in water.

FIG. 3: shows release rate curves of the first batch of topiramate sustained-release coating pellets of Example 6 in different release media.

FIG. 4: shows release rate curves of the first batch of topiramate sustained-release coating pellets of Example 6 under different rotation speed conditions.

FIG. 5: shows in vivo blood concentration curve of topiramate sustained-release coating pellets.

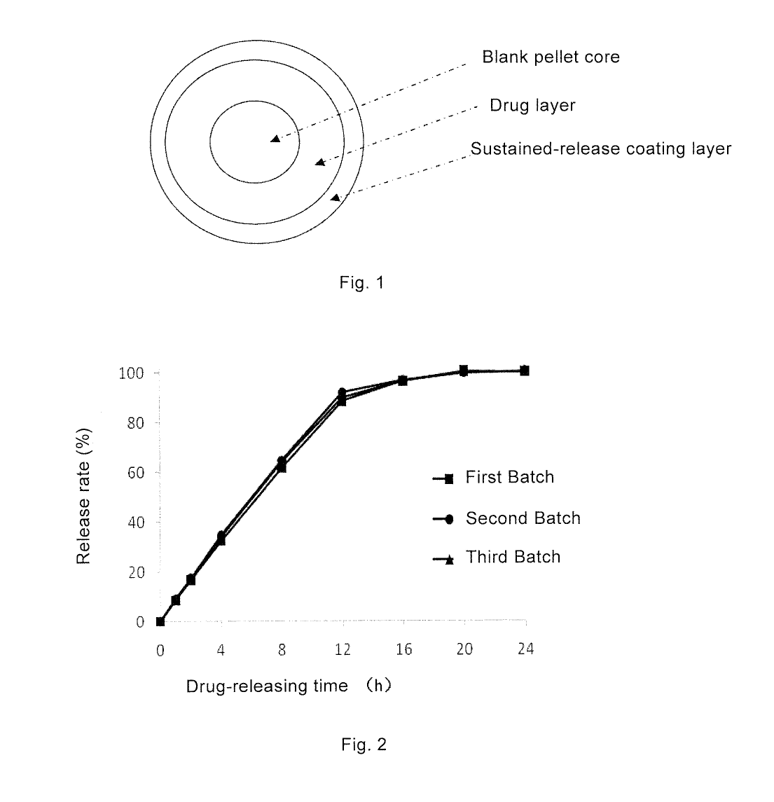

### SPECIFIC MODELS FOR CARRYING OUT THE INVENTION

The present invention is further illustrated with the following specific examples. It should be understood that the following examples are merely used to illustrate the present invention, but do not intend to limit the scope of the present invention. If specific conditions are not given in the examples, conventional conditions or conditions suggested by manufacturers were used. If the manufacturers of the used reagents or instruments were not given, they were all conventional products commercially available in markets.

In the following examples, unless specifically pointed out, the obtained parameters were all calculated according to the following formulations:

Pellet drug-loading rate (%)=(W_{total weight of pellets}−W_{blank pellet core weight})/W_{amount of bulk drug}×100%

Weight increment of sustained-release coating (%)=(W_{total weight of pellets after sustained-release coating}−W_{total weight of pellets before sustained-release coating})/W_{total weight of pellets after sustained-release coating}×100%

Adhesion rate of pellet=(W_{total weight of pellets after coating}−W_{total weight of pellets without adhesion})/W_{total weight of pellets after coating}×100%

In the examples of the present invention, unless specifically pointed out, release rates of topiramate were all measured by the following method. According to the first method (for sustained-release preparation or controlled-release preparation) of Release Rate Measurement (Appendix X D) of Part II of Chinese Pharmacopoeia, 2010 Edition, the apparatus as stated in the second method (slurry method) of Dissolution Rate Measurement (Appendix X C) of Part II of Chinese Pharmacopoeia, 2010 Edition, was used to perform the measurement using water (500 ml) as releasing media, at 37° C. and rotation speed of 100 rpm. Samples (5 ml, supplemented with equivalent volume of media at the meantime) were taken at specified time points and filtrated, the subsequent filtrates were used as test solutions. High performance liquid chromatography (Appendix V D of Part II of Chinese Pharmacopoeia, 2010 Edition) was used, octylsilane-bonded silica gel was used as packing agent, column temperature was 35° C., 50% methanol was mobile phase, differential refractive detector was used, flow rate was 1.5 ml per minute. 200 μl of test solution was taken, injected in liquid chromatograph, the peak area of topiramate as main drug was recorded; topiramate was separately taken as control sample and measured by the same method, and accumulative release percentages of drug at different time points were calculated by external standard method.

### Example 1

### Comparison of Drug-Loading and Coating Between Drug-Containing Solutions with and without Binding Agent

Prescription:

| Pre- | | Binding agent (g) | | | | |
|---|---|---|---|---|---|---|
| scrip- | | | | | Free of bind- | 50% etha- |
| tion | Topiramate (g) | HPMC | PVP | HPC | ing agent | nol (ml) |
| 1 | 230 | 6.9 g | — | — | — | 1150 |
| 2 | 230 | — | 6.9 g | — | — | 1150 |
| 3 | 230 | — | — | 6.9 g | — | 1150 |
| 4 | 230 | — | — | — | — | 1150 |

Preparation Method:

4 Parts of topiramate raw material were weighed, 230 g per part, separately added with suitable amount of 50% ethanol, stirred under heating at 40° C.-50° C. for dissolution; then HPMC(E5), PVP K30 and HPC, each 6.9 g, were separately weighed, added in order to the first part, the second part, and the third part solutions, while the fourth part was free of binding agent; they were stirred and heated at 40° C.-50° C. for dissolution, then added with 50% ethanol to reach 1150 ml to obtain drug-containing coating solutions with different binding agents.

500 g of sucrose pellet cores (710-850 μm) were placed in a fluidized bed bottom spray coating pan, the inlet air temperature was set as 55° C. (to keep pan internal temperature at 40±2° C.); inlet air pressure was 0.35 bar; atomization pressure was 1.5 bar; solution spray rate was 5-15 g/min (regulated according to fluidization state at any time). The drug-containing coating solution was sprayed in manner of bottom spray on surface of blank pellet cores when the sucrose pellet cores were in fluidization state, after the end of drug-loading, the material was continuously fluidized at 45° C. for 5 min to obtain drug-loading pellets with different binding agents, which were weighed, and results were shown in Table 1.

TABLE 1
Results of drug-loading and coating using drug-containing
solutions with and without a binding agent

| | Prescrip- | Prescrip- | Prescrip- | Prescrip- |
|---|---|---|---|---|
| Index | tion 1 | tion 2 | tion 3 | tion 4 |
| Adhesion degree (%) | 7.8 | 3.7 | 5.5 | 2.1 |
| Drug-loading time (min) | 71 | 62 | 68 | 54 |
| Drug-loading rate of | 94.2 | 93.7 | 95.1 | 95.4 |
| pellet(%) | | | | |

The results showed that the sustained-release pharmaceutical composition of topiramate without binding agent in the present invention had short coating and drug-loading time, and high drug-loading rate.

### Example 2

### Results of Drug-Loading and Coating Using Drug-Containing Coating Solutions with Different Solvents

4 Parts of topiramate raw material were weighed, 230 g per part, separately added with suitable amount of 50% ethanol, 70% ethanol, 95% ethanol, and anhydrous ethanol, stirred and heated at 40° C.-50° C. for dissolution; then corresponding solvent was supplemented to reach 1150 ml to obtain drug-containing coating solutions with different solvents as dissolvent.

500 g of sucrose pellet cores (710-850 μm) were placed in a fluidized bed bottom spray coating pan, the inlet air temperature was set as 55° C. (to keep pan internal temperature at 40±2° C.); inlet air pressure was 0.35 bar; atomization pressure was 1.5 bar; solution spray rate was 5-15 g/min (regulated according to fluidization state at any time). The drug-containing coating solution with different solvents as dissolvent was sprayed on surface of blank pellet cores in manner of bottom spray when the sucrose pellet cores were in fluidization state, after the end of drug-loading, the material was continuously fluidized at 45° C. for 5 min to obtain drug-loading pellets with different solvents as dissolvent, which were weighed, and results were shown in Table 2.

TABLE 2
Drug-loading and coating results of drug-containing coating
solutions with different solvents as dissolvent

| | 50% | 70% | 95% | Anhydrous |
|---|---|---|---|---|
| Index | ethanol | ethanol | ethanol | ethanol |
| Adhesion degree (%) | 2.1 | 2.2 | 1.9 | 1.8 |
| Drug-loading time(min) | 54 | 50 | 48 | 44 |
| Drug-loading rate on | 95.4 | 94.5 | 91.6 | 90.4 |
| pellet (%) | | | | |

### Example 3

### Comparison of Release Rates of Sustained-Release Pellets of Topiramate with Blank Pellet Cores Having Different Particle Diameters

Prescription

1) Prescription of drug-loading pellet:

Blank pellet core

| | Topiramate | Range of particle | Weight |
|---|---|---|---|
| Prescription | (g) | diameter (μm) | (g) |
| 5 | 230 | 300-400 | 500 |
| 6 | 230 | 500-610 | 500 |
| 7 | 230 | 610-750 | 500 |

2) Prescription of sustained-release coating layer:

| Prescription | Ethyl cellulose (g) | PVP K30 (g) |
|---|---|---|
| 5 | 50 | 16.5 |
| 6 | 40 | 13.2 |
| 7 | 30 | 10.0 |

Preparation Method:

(1) 230 g of topiramate raw material was weighed, added with a suitable amount of 50% ethanol, stirred and heated at 40-50° C. for dissolution, added 50% ethanol to reach 1150 ml to obtain a drug-containing coating solution.

300 μm-400 μm, 500 μm-610 μm, 710 μm-850 μm sucrose pellet cores were separately weighed, each 500 g, placed in a fluidized bed bottom spray coating pan, the inlet air temperature was set as 55° C. (to keep pan internal temperature at 40±2° C.); inlet air pressure was 0.35 bar; atomization pressure was 1.5 bar; solution spray rate was 5-15 g/min (regulated according to fluidization state at any time). The drug-containing coating solution was sprayed in manner of bottom spray on surface of blank pellet cores when the sucrose pellet cores were in fluidization state, after the end of drug-loading, the material was continuously fluidized at 45° C. for 5 min to obtain drug-loading pellets of topiramate.

(2) The prescription amount of ethyl cellulose (EC) was weighed, added with a suitable amount of 95% ethanol for dissolution, then added with the prescription amount of PVP K30, dissolved to obtain a sustained-release coating solution.

(3) The above drug-loading pellets of topiramate with different particle diameters were weighed, each 500 g, and separately placed in a fluidized bed bottom spray coating pan, the inlet air temperature was set as 40-45° C. (to keep pan internal temperature at 30-35° C.); inlet air pressure was 0.35 bar; atomization pressure was 1.5 bar, solution spray rate was 3-12 g/min. The sustained-release coating solutions of the 3 prescriptions were separately sprayed in manner of bottom spray on surfaces of drug-loading pellets with different particle diameters when the drug-loading pellets were in fluidization state, to obtain sustained-release pellets of topiramate with different particle diameters, in which the weight increments of sustained-release coating were 10.9%, 8.8% and 6.7%, respectively. According to calculation, the adhesion degrees of pellets were separately 2.2%, 2.1%, 1.8%.

The measurement results of drug release rates of the prepared sustained-release pellets of topiramate were shown in Table 3.

TABLE 3
Evaluation results of release rates of pellets
with different particle diameters

| Pre- | | | | | | | |
|---|---|---|---|---|---|---|---|
| scrip- | Particle diameters | Release rate (%) | | | | | |
| tion | of blank pellet cores | 1 h | 4 h | 8 h | 12 h | 16 h | 20 h |
| 5 | 300-400 μm | 21.6 | 55.5 | 88.1 | 98.5 | 100.4 | 101.2 |
| 6 | 500-610 μm | 16.7 | 49.5 | 80.6 | 92.4 | 98.5 | 99.6 |
| 7 | 610-750 μm | 19.4 | 56.8 | 85.4 | 96.2 | 99.8 | 100.4 |

### Example 4

### Comparison of Release Rates of Sustained-Release Pellets of Topiramate Coated with Different Types of Sustained-Release Materials

Prescription of Sustained-Release Coating:

| Prescription | 8 | 9 | 10 |
|---|---|---|---|
| Drug-loading | 500 g | 500 g | 500 g |
| pellets | | | |
| Eudragit RS30D | 133 g(corre- | — | — |
| | sponding to 40 | | |
| | g of dry resin) | | |
| Eudragit NE30D | — | 167 g (corre- | — |
| | | sponding to 50 | |
| | | g of dry resin) | |
| Eudragit RL30D | — | — | 200 g(corre- |
| | | | sponding to 60 |
| | | | g of dry resin) |
| Talc powder | 20 | 25 | 30 |
| Water | 246 | 309 | 370 |

Preparation Method:

Aqueous dispersions of Eudragit RS30D, Eudragit NE 30D, Eudragit RL30D in prescription amounts were separately weighed, added with water in 1 time amount, stirred homogeneously; the talc powder in prescription amount was added to the residual water, homogenized with a high-shear homogenizer for 3 min, the obtained suspension was slowly poured into the above aqueous dispersions, stirred homogeneously, passed through 80 mesh sieve, to obtain sustained-release coating solutions.

500 g of drug-loading pellets as prepared according to Prescription 4 of Example 1 was placed in a fluidized bed bottom spray coating pan, the inlet air temperature was set as 25-30° C. (to keep pan internal temperature at 23-25° C.); inlet air pressure was 0.35 bar; atomization pressure was 1.5 bar; solution spray rate was 3-5 g/min. The above 3 sustained-release coating solutions were separately sprayed on surface of the drug-loading pellets in manner of bottom spray when the drug-loading pellets were in fluidization state, to obtain 3 kinds of sustained-release pellets respectively with Eudragit RS30D, Eudragit NE 30D, and Eudragit RL30D as sustained-release coating material, and the weight increments of sustained-release coating were separately 9.7%, 11.9%, and 13.9%. The obtained sustained-release pellets of topiramate were subjected to aging and heating treatment in a high temperature oven at 40° C. for 24 h. According to calculation, the adhesion degrees of pellets were 2.8%, 2.7%, 3.0%, respectively.

The measurement results of drug release rates were shown in Table 4.

TABLE 4
Evaluation results of release rates of sustained-release
pellets of topiramate with different sustained-release coating materials

| | Release rate (%) | | | | | |
|---|---|---|---|---|---|---|
| Prescription | 1 h | 4 h | 8 h | 12 h | 16 h | 20 h |
| 8 | 19.6 | 44.6 | 70.2 | 88.3 | 97.7 | 99.7 |
| 9 | 15.5 | 34.4 | 66.3 | 77.9 | 92.2 | 94.8 |
| 10 | 16.4 | 36.5 | 68.3 | 79.4 | 95.1 | 98.5 |

### Example 5

### Preparation of Sustained-Release Pellets of Topiramate Prescription of Sustained-Release Coating

| Prescription | 11 | 12 | 13 |
|---|---|---|---|
| EC | 30 g | 30 g | 30 g |
| PVP K30 | 9 g | 9.6 g | 10.5 g |

Preparation Method:

According to the amount proportions of the above prescriptions, ethyl cellulose was dissolved with a suitable amount of 95% ethanol, then separately added with proportion amounts of PVP K30 and dissolved to obtain sustained-release coating solutions.

500 g of drug-loading pellets as prepared with the drug-containing coating solution without a binding agent of Example 1 was placed in a fluidized bed bottom spray coating pan, the inlet air temperature was set as 40-45° C. (to keep pan internal temperature at 30-35° C.); inlet air pressure was 0.35 bar; atomization pressure was 1.5 bar; solution spray rate was 3-12 g/min. The sustained-release coating solutions with different proportions of ethyl cellulose and PVP K30 were separately sprayed on surface of the drug-loading pellets in manner of bottom spray when the drug-loading pellets were in fluidization state, the weight increments of sustained-release coating were separately 6.56%, 6.65%, and 6.79%, so as to obtain sustained-release pellets of topiramate with different proportions of ethyl cellulose and PVP K30. According to calculation, the adhesion degrees of pellets were 2.3%, 2.4% and 2.1%, respectively.

The measurement results of drug release rate were shown in Table 5.

TABLE 5
Evaluation results of release rates of sustained-release pellet
of topiramate as prepared according to the prescription 11-13

| | Release rate (%) | | | | | |
|---|---|---|---|---|---|---|
| Prescription | 1 h | 4 h | 8 h | 12 h | 16 h | 20 h |
| 11 | 17.4 | 36.6 | 64.3 | 82.5 | 90.7 | 95.5 |
| 12 | 21.8 | 47.6 | 76.4 | 91.3 | 95.7 | 97.8 |
| 13 | 24.2 | 51.4 | 83.8 | 99.7 | 99.6 | 100.2 |

### Example 6

### Experiment of Process Repeatability

(1) Preparation of Topiramate Drug-Loading Pellet Cores without a Binding Agent

276 g of Topiramate raw material was weighed, added with a suitable amount of 70% ethanol, stirred under heating at 40-50° C., dissolved, added with 70% ethanol to reach 1380 ml, to obtain a drug-containing coating solution.

600 g of 710 μm-850 μm sucrose pellet cores was weighed and placed in a fluidized bed bottom spray coating pan, the inlet air temperature was set as 55° C. (to keep pan internal temperature at 40±2° C.); inlet air pressure was 0.35 bar; atomization pressure was 1.5 bar; solution spray rate was 5-15 g/min (regulated according to fluidization state at any time). The drug-containing coating solution was sprayed on surface of blank sucrose pellet cores in manner of bottom spray when the sucrose pellet cores were in fluidization state. After the end of drug-loading, the material was continuously fluidized at 45° C. for 5 min to obtain topiramate drug-loading pellet cores without a binding agent, which were weighed, the total weight Wₜₒₜₐₗ of the pellets after the end of drug-loading was recorded, and the drug-loading rate and product yield of the pellets were calculated and shown in Table 6.

TABLE 6
Results of process repeatability of topiramate drug-loading pellet cores without binding agent

| | Production scale | Amount of the | Amount of | Amount of the | | |
|---|---|---|---|---|---|---|
| Sample | (preparation | charged main | sucrose pellet | produced drug-loading | Drug-load- | Product |
| batch | unit/batch) | drug (g/batch) | cores (g/batch) | pellet cores (g/batch) | ing rate (%) | yield (%) |
| 1 | 12000 | 276 | 600 | 864 | 95.7 | 98.6 |
| 2 | 12000 | 276 | 600 | 862 | 94.9 | 98.4 |
| 3 | 12000 | 276 | 600 | 863 | 95.3 | 98.5 |

Notation: the dose of topiramate of each preparation unit was expressed as 23 mg; the product yield was calculated by dividing the amount of drug-loading pellet cores by the total amount of the charged raw materials and adjuvants.

(2) Preparation of Sustained-Release Coating Pellet of Topiramate

48 g of Ethyl cellulose was weighed, added with a suitable amount of 95% ethanol, stirred under heating at 40° C.-50° C., dissolved, then added with about 16.2 g of PVP K30, stirred under heating at 40° C.-50° C., dissolved, stirred homogenously, added with 95% ethanol to reach 1152 ml, to obtain a sustained-release coating solution.

800 g of drug-loading pellets as above prepared was placed in a fluidized bed bottom spray coating pan, the inlet air temperature was set as 40-45° C. (to keep pan internal temperature at 30-35° C.); inlet air pressure was 0.35 bar; atomization pressure was 1.5 bar; solution spray rate was 3-12 g/min. The sustained-release coating solution was sprayed on surface of the drug-loading pellets in manner of bottom spray when the drug-loading pellets were in fluidization state, to obtain 3 batches of sustained-release pellets of topiramate, their weight increments of sustained-release coating were separately 6.87%, 6.98%, and 7.08%. According to calculation, the adhesion degrees of pellets were 2.1%, 2.0% and 2.1%, respectively. The results were shown in FIG. 2 and Table 7.

TABLE 7
Experimental results of process repeatability of sustained-release coating pellets of topiramate

| | | | | | Sustained- | | | | | |
|---|---|---|---|---|---|---|---|---|---|---|
| | Product | Amount of | Amount | Amount | release | | | | | |
| | amount | drug-loading | of ethyl | of PVP | pellet of | | | | | |
| Sample | (preparation | pellet cores, | cellulose, | K30, | topiramate, | Product | Release rate (%) | | | |
| batch | unit/batch) | g/batch | g/batch | g/batch | g/batch | yield (%) | 1 h | 4 h | 8 h | 16 h |
| 1 | 10000 | 800 | 48 | 16.2 | 859 | 99.3 | 14.4 | 37.2 | 68.5 | 96.4 |
| 2 | 10000 | 800 | 48 | 16.2 | 860 | 99.5 | 15.1 | 38.6 | 69.5 | 96.8 |
| 3 | 10000 | 800 | 48 | 16.2 | 861 | 99.6 | 15.9 | 39.1 | 68.8 | 96.4 |

The results showed that the sustained-release pharmaceutical composition of topiramate (coating pellets) of the present invention had good process repeatability.

### Example 7

### Effects of Different Dissolution Media on Release Rate of Sustained-Release Pellet of Topiramate

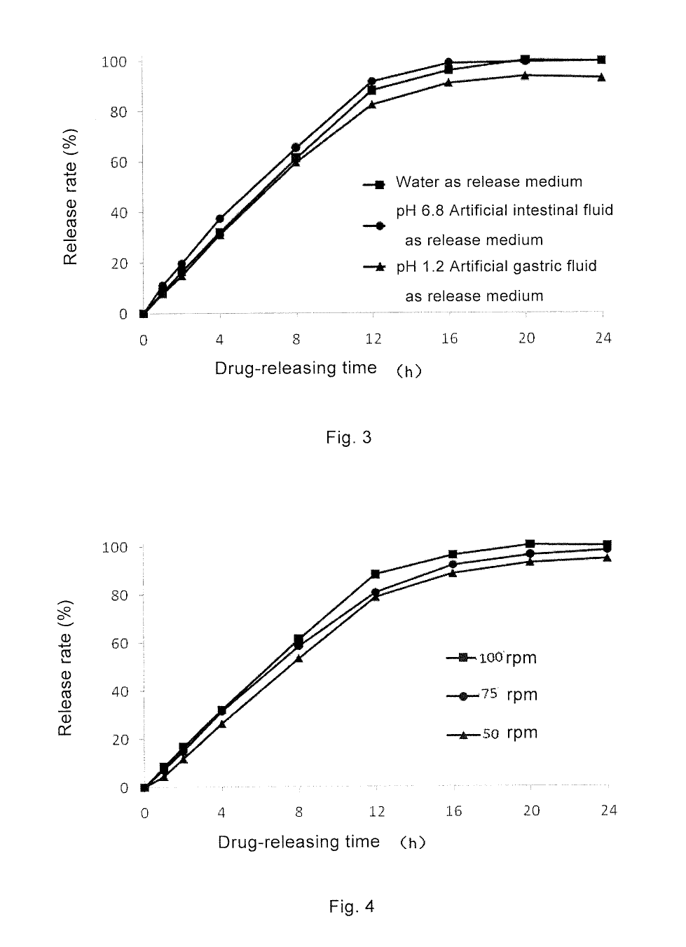

In order to verify whether acidic, basic solvent media would influence the release rate of the sustained-release pellets of the present invention, 0.1 mol/L HCl (pH1.2) was prepared as artificial gastric fluid, 0.2 ml/L phosphate buffer (pH6.8) was prepared as artificial intestinal fluid, and these media and water (500 ml) were used as release media, rotation speed was 100 rpm, 37° C. Samples (5 ml, supplemented with equivalent volume of media at the meantime) were taken at 1, 2, 4, 8, 12, 16, 20, 24 h, filtrated, and the subsequent filtrates were used as test solutions. High performance liquid chromatography (Appendix V D of Part II of Chinese Pharmacopoeia, 2010 Edition) was used, octylsilane-bonded silica gel was used as packing agent, 50% methanol was mobile phase, differential refractive detector was used, flow rate was 1.5 ml per minute. 200 μl of test solution was taken, injected in liquid chromatograph, the peak area of topiramate as main drug was recorded; topiramate was separately taken as control sample and measured by the same method, and accumulative release percentages of drug at different time points were calculated by external standard method. The release profile of sample of Batch 1 in Example 6 (2) in the above media were drawn, and the results were shown in FIG. 3.

The results showed that the drug release profiles of the sustained-release pellet of topiramate in the artificial gastric fluid, water and the artificial intestinal fluid were substantially consistent (since topiramate was unstable in pH1.2 artificial gastric fluid and had degradation reaction, the release rate in the artificial gastric fluid in the present experiment was derived from the sum of main drug topiramate and degradation products), which suggested that the product could release drug consistently in different sites of gastrointestinal tract, so as to ensure stable pharmacological effects of topiramate as active ingredient.

### Example 8

### Effects of Different Rotation Speeds on Release Rate of Sustained-Release Pellet of Topiramate

In order to verify whether gastrointestinal motility would influence the release rate of the sustained-release pellets of the present invention, rotation speed was set as 50 rpm, 75 rpm and 100 rpm, respectively, and water (500 ml) were used as release media, 37° C. Samples (5 ml, supplemented with equivalent volume of media at the meantime) were taken at 1, 2, 4, 8, 12, 16, 20, 24 h, filtrated, and the subsequent filtrates were used as test solutions. High performance liquid chromatography (Appendix V D of Part II of Chinese Pharmacopoeia, 2010 Edition) was used, octylsilane-bonded silica gel was used as packing agent, 50% methanol was mobile phase, differential refractive detector was used, flow rate was 1.5 ml per minute. 200 μl of test solution was taken, injected in liquid chromatograph, the peak area of topiramate as main drug was recorded; topiramate was separately taken as control sample and measured by the same method, and accumulative release percentages of drug at different time points were calculated by external standard method. The release profile of sample of Batch 1 in Example 6 (2) under the above different rotation speeds were drawn, and the results were shown in FIG. 4.

The results showed that the drug release profiles of the sustained-release pellet of topiramate under rotation speed ranging 50-100 rpm were substantially consistent, which suggested that the product could release drug consistently under different situations of gastrointestinal motility, so as to ensure stable pharmacological effects of topiramate as active ingredient.

### Example 9

### Studying on Drug Release Consistency (1)

The prescriptions 8-10 were repeated 3 times according to the method of Example 4, and their drug release consistency was considered. The results were shown in Table 8.

TABLE 8
Results of drug release consistency of sustained-release pellet of topiramate
(mean ± standard deviation)

| | Drug release rate at different time points (%) (mean ± standard deviation) | | | | |
|---|---|---|---|---|---|
| Prescription | 1 h | 4 h | 8 h | 16 h | 20 h |
| Prescription 8 | 19.6 ± 8.7 | 51.2 ± 8.3 | 75.2 ± 9.8 | 93.3 ± 9.1 | 95.7 ± 5.2 |
| Prescription 9 | 15.1 ± 7.1 | 38.8 ± 7.4 | 68.7 ± 8.7 | 92.9 ± 9.8 | 95.3 ± 4.1 |
| Prescription 10 | 14.4 ± 8.8 | 36.5 ± 8.3 | 65.3 ± 9.6 | 92.4 ± 8.1 | 95.1 ± 5.2 |
| Example 6 | 14.4 ± 2.5 | 37.2 ± 1.8 | 68.5 ± 1.4 | 96.4 ± 1.2 | 100.2 ± 0.9 |

The results showed that the sustained-release pharmaceutical composition of topiramate (coating pellets) of the present invention had good drug release consistency.

### Example 10

### Studying on Drug Release Consistency (2)

Prescription:

Drug layer

| Prescrip- | Topiramate | Sodium dodecyl | Tween | Talc |
|---|---|---|---|---|
| tion | (g) | sulfate (g) | 80 (g) | powder (g) |
| 14 | 230 | 3.45 | — | — |
| 15 | 230 | — | 6.90 | — |
| 16 | 230 | — | — | 11.5 |

Sustained release layer

| Name | Amount |
|---|---|
| EC (g) | 30 |
| PVP K30 (g) | 10.0 |
| Aerosil (g) | 3.0 |
| 95% ethanol (ml) | 720 |

Preparation Method:

(1) The ingredients of drug layer in the above prescription amounts were weighed, added with a suitable amount of 50% ethanol, stirred under heating at 40° C.-50° C., dissolved (prescriptions 14 and 15) or suspended (prescription 16), added with 50% ethanol to reach 1150 ml, to obtain drug-containing coating solutions (prescriptions 14, 15) or suspension (prescription 16).

500 g of 610 μm-750 μm sucrose pellet cores was weighed and placed in a fluidized bed bottom spray coating pan, the inlet air temperature was set as 55° C. (to keep pan internal temperature at 40±2° C.); inlet air pressure was 0.35 bar; atomization pressure was 1.5 bar; solution spray rate was 5-15 g/min (regulated according to fluidization state at any time). The drug-containing coating solutions or suspension were sprayed on surface of blank pellet cores in manner of bottom spray when the sucrose pellet cores were in fluidization state. After the end of drug-loading, the material was continuously fluidized at 45° C. for 5 min to obtain topiramate drug-loading pellets.

(2) Ethyl cellulose (EC) in prescription amount was weighed, added with 95% ethanol, stirred under heating at 40° C.-50° C., dissolved, then added with the prescription amount of PVP K30, stirred under heating at 40° C.-50° C., dissolved, stirred homogeneously, added with the prescription amount of aerosil, added 95% ethanol to the prescription amount, stirred, to obtain sustained-release coating solution.

500 g of the above topiramate drug-loading pellet cores was separately weighed and placed in a fluidized bed bottom spray coating pan, the inlet air temperature was set as 40-45° C. (to keep pan internal temperature at 30-35° C.); inlet air pressure was 0.35 bar; atomization pressure was 1.5 bar, solution spray rate was 3-12 g/min. The sustained-release coating solutions of the 3 prescriptions were sprayed on surface of drug-loading pellets with different corresponding particle diameters in manner of bottom spray when the drug-loading pellets were in fluidization state, to obtain sustained-release pellets of topiramate with different particle diameters, the weight increment of coating was 6%. Via calculation, the adhesion degrees of pellets were 1.9, 2.0, 1.7%, respectively. The measurement results of release rates were shown in Table 9.

TABLE 9
Measurement results of drug release rates of prescriptions 14-16

| | Release rate (%) | | | | |
|---|---|---|---|---|---|
| Prescription | 1 h | 4 h | 8 h | 16 h | 20 h |
| Prescription 14 | 9.60 | 39.6 | 66.3 | 95.7 | 97.1 |
| Prescription 15 | 9.4 | 36.6 | 65.3 | 96.8 | 98.8 |
| Prescription 16 | 9.2 | 37.8 | 68.2 | 96.9 | 99.7 |

The results showed that the sustained-release pharmaceutical composition of topiramate (coating pellets) of the present invention good drug release consistency.

### Example 11

### Preparation of Sustained-Release Pellets of Topiramate

500 g of the topiramate drug-loading pellet cores prepared according to Prescription 1 in Example 1, which drug layer contained binding agent HPMC, was weighed, and placed in a fluidized bed bottom spray coating pan. 720 ml of 95% ethanol was used to prepare a sustained-release coating solution containing ethyl cellulose and PVP K30 respectively in amount of 30 g and 10 g. The inlet air temperature was set as 40-45° C. (to keep pan internal temperature at 30-35° C.); inlet air pressure was 0.35 bar; atomization pressure was 1.5 bar; solution spray rate was 3-12 g/min. The sustained-release coating solution was sprayed on surface of drug-loading pellets in manner of bottom spray when the drug-loading pellets were in fluidization state, to obtain sustained-release pellets of topiramate with a drug layer containing binding agent HPMC, the weight increment of coating was 6.86.

### Example 12

### Preparation of Sustained-Release Pellets of Topiramate

Prescription

| | Dosage per 10000 | Dosage ratio |
|---|---|---|
| Name of ingredients | preparation units (g) | (%)(w/w) |
| Topiramate | 230 | 36.85 |
| Microcrystalline cellulose | 324.9 | 52.05 |
| (Avicel ® PH102) | | |
| Methyl cellulose | 20.1 | 3.22 |
| (Methocel ™A15LV) | | |
| Ethyl cellulose | 34.14 | 5.47 |
| Povidone (Povidone K30) | 14.92 | 2.39 |

Preparation Method:

Topiramate as main drug and microcrystalline cellulose as filling agent in the prescription amounts passed through sieve and mixed homogeneously; 70% ethanol was used to dissolve methyl cellulose (Methocel™A15LV) to obtain a solution as binding agent with a suitable concentration, and then used to form soft material; the soft material was placed in an extruder using a certain mesh sieve and at a extrusion rate to extrude rod like granules; the extruded granules were placed in a spheronizator and spheronized under certain spheronization speed for 3-5 min, the obtained pellets were dried in 40° C. oven for 2 h to obtain topiramate drug-loading pellet cores.

The prescription amounts of ethyl cellulose and Povidone (Povidone K30) were weighed, added with 820 ml of 95% ethanol, stirred and dissolved to form a sustained-release coating solution. The above prepared topiramate drug-loading pellet cores were placed in a fluidized bed bottom spray coating pan, the inlet air temperature was set as 40-45° C. (to keep pan internal temperature at 30-35° C.); inlet air pressure was 0.35 bar; atomization pressure was 1.5 bar; solution spray rate was 1-3 g/min. The sustained-release coating solutions was sprayed on surface of drug-loading pellet cores when the drug-loading pellets were in fluidization state, to obtain sustained-release pellets of topiramate, the weight increment of coating was 6.68%.

### Example 13

### Studying on Stability of Topiramate Pellets

The sustained-release pellets of topiramate prepared in Example 11 and Example 12, and the first Batch of sustained-release pellets of topiramate prepared in Example 6 were separately placed nakedly in sealed dryer with saturated NaCl solution, then the dryer was placed in high temperature 60° C. oven, acceleration conditions of high temperature and high humidity (60° C., RH75%) were set in the meantime, and samples were taken on 0^{th}, 5^{th} and 10^{th} day.

The content of pellet sample was poured out, placed in 10 mL volumetric flask, dissolved with a suitable amount of methanol under ultrasonic waves, then diluted with water in 5 times volume to reach scale, so that the concentration of main drug was about 5 mg/ml, 0.45 μm organic microfiltration membrane was used for filtration, primary filtrate was discarded, the subsequent filtrate were used as test solutions. High performance liquid chromatography (Appendix V D of Part II of Chinese Pharmacopoeia, 2010 Edition) was used, octylsilane-bonded silica gel was used as packing agent, column temperature was 35° C., 40% methanol was mobile phase, differential refractive detector was used, flow rate was 1.5 ml per minute. 200 μl of the test solution was taken, injected in liquid chromatograph, chromatogram was recorded until 3 times the time period of main peak retention time, if the test solution had peaks of impurities, the total content of impurities was calculated by peak area normalization method, and the results were shown in Table 10. It could be seen that the sustained-release composition of topiramate (the coating type drug-loading pellet cores in which the topiramate drug layer was free of binding agent, sample of Example 6) as disclosed in the present invention had stability superior to the matrix type drug-loading pellet cores (sample of Example 12) and the coating type drug-loading pellet cores (sample of Example 11) which all had topiramate drug layer containing a binding agent.

TABLE 10
Results of stability of sustained-release pellets of topiramate

| | Degradation products | | |
|---|---|---|---|
| | (relevant substances) (%) | | |
| Sample of stability | 0^{th} day | 5^{th} day | 10^{th} day |
| Sample of Example 11 | 0.15 | 0.37 | 0.55 |
| Sample of Example 12 | 0.15 | 0.38 | 0.57 |
| Sample of Example 6 | 0.15 | 0.21 | 0.28 |

The results showed that the sustained-release pharmaceutical composition of topiramate of the present invention had good stability.

### Example 14

### Studying on In Vivo Pharmacokinetics of Sustained-Release Pellet of Topiramate

Test samples: the topiramate drug-loading pellet core (rapid-release pellet) as prepared in Example 6 was used as reference preparation, and the sustained-release pellet of topiramate as prepared in Example 6 was used as test preparation. Administration dose was all 23 mg expressed as topiramate (main drug).

Test subjects: 6 Beagles, half male and half female, the body weight of Beagles was 8.97±1.05 kg.

Dosage regimen: 6 Beagles were subjected to double cycle random crossover test design, separately orally administrated once with equivalent dose of the test preparation containing 23 mg of topiramate as main drug and the reference preparation containing 23 mg of topiramate as main drug, an wash-out period with interval time of 15 days was set between the two cycles. Blood samples (2 mL) were separated taken from leg veins of the Beagles at 0.5, 1, 2, 3, 4, 6, 8, 10, 12, 16, 24, 36, 48 h after administration, placed in negative pressure glass tubes treated with heparin sodium, centrifuged at 4000 r/min for 10 min to separate plasma, the plasma was removed to 1 mL EP tube, labeled with test number, random number of Beagle and blood sampling time, the blood samples were kept at −20° C. for treatment and analysis.

Plasma sample treatment: 100 μL of plasma of Beagle after administration was taken, placed in 1.5 ml centrifuge tube, added with 20 μl of water, added with 20p1 of internal standard solution (500 ng/ml Nimesulide solution), added with 0.5 ml of methanol as precipitator, subjected to eddy for 3 min, centrifuged for 10 min (9500 rpm), supernatants (20 μl) were separately sucked up, and analyzed with LC/MS/MS under the following chromatography conditions, and chromatograms were recorded.

Chromatography conditions: analysis column was Zorbax C8, 5 μm particle size, 150×4.6 mm I.D., Agilent Company of US; pre-column was C18 protection column, 4×3.0 mm I.D., Phenomenex Company, USA; column temperature was 25° C.; mobile phase was methanol-0.5 mM ammonium acetate (75:25, v/v); flow rate was 0.5 mL/min; internal standard was Nimesulide (500 ng/mL).

Mass spectrometric conditions: API 3000 type tandem quadrupole mass spectrometer. Ion source was atmospheric chemical ion source (Turbo Ionspray source); detection was performed in negative ion manner; ejection voltage was −4200 V; source temperature was 450° C.; nebulizer gas (NEB) was 8; curtain gas (CUR) was 11; collision gas (CAD) was 5; scanning manner was multiple reaction monitoring (MRM), the ion reactions for quantitative analysis were separately: m/z 338→m/z 78 (topiramater, CE −55 V), m/z 307→m/z 229 (internal standard nimesulide, CE −20 V); scanning time was 150 msec.

Pharmacokinetic data treatment: blood concentration data were analyzed with DAS 2.0 analytic software.

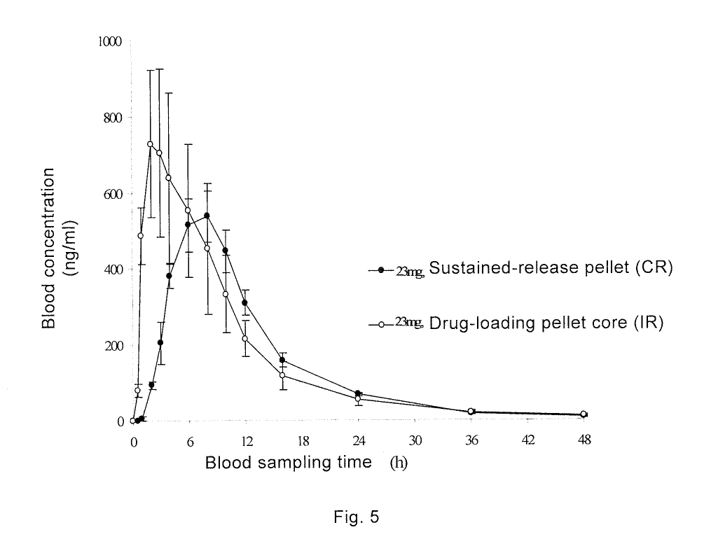

Results of measurement: after Beagles were orally administered with equivalent dose (23 mg) of reference preparation (first batch of topiramate drug-loading pellet cores of Example 6) and test preparation (first batch of sustained-release pellet of topiramate of Example 6), average blood concentrations (pg/ml) at different time points were shown in FIG. 5, and main pharmacokinetic parameters were shown in Table 11.

TABLE 11
Main pharmacokinetic parameters

| | | | | Relative |
|---|---|---|---|---|
| | Tₘₐₓ | Cₘₐₓ | AUC_{0→Tn} | bioavail- |
| Test sample | (h) | (μg · ml⁻¹) | (h · μg · ml⁻¹) | ability (%) |
| Reference | 2.33 ± 0.47 | 1.11 ± 0.13 | 9.39 ± 3.23 | 100.00% |
| preparation | | | | |
| Test | 7.33 ± 0.94 | 0.55 ± 0.07 | 8.72 ± 2.67 | 92.87% |
| preparation | | | | |

The results of FIG. 5 and Table 11 showed that the Beagles orally administered with the sustained-release composition of topiramate as provided by the present invention (first batch of sustained-release pellets of topiramate of Example 6, containing 23 mg of topiramate) showed significantly extended Tmax, significantly decreased Cmax, in comparison with the rapid-release topiramate drug-loading pellet cores (reference preparation, first batch of topiramate pellet cores of Example 1, containing 23 mg of topiramate), and more important, the relative bioavailability of the test preparation was 92.87% of that of the reference preparation. This indicated that the topiramate sustained-release composition as provided by the present invention had biologically equivalent to the reference preparation, and showed significant profiles of sustained-release preparation, that was, the peak concentration decreased significantly, and the action time was significantly extended.

Although the specific models of the present invention have been described in details, those skilled in the art would understand that these details can be modified or changed according to all teachings of disclosures in the art, and all these changes fall into the protection scope of the present invention. The total scope of the present invention is given by the appended claims and any equivalents thereof.

What is claimed is:

1. A sustained-release pharmaceutical composition of topiramate, in which drug layer is free of binding agent.

2. The sustained-release pharmaceutical composition of topiramate according to claim 1, which is topiramate sustained-release pellet, and the topiramate sustained-release pellet comprises a blank pellet core, a drug layer, and a sustained-release coating layer.

3. The sustained-release pharmaceutical composition of topiramate according to claim 1 or 2, wherein topiramate is in an amount of 10%-50%, preferably 15%-45%, more preferably 20%-40%, relative to total weight of the composition.

4. The sustained-release pharmaceutical composition of topiramate according to claim 2, wherein the blank pellet core has a particle diameter of 150 μm-1500 μm, preferably 300 μm-1000 μm, more preferably 400 μm-850 μm, further preferably 610 μm-750 μm.

5. The sustained-release pharmaceutical composition of topiramate according to claim 2, wherein the sustained-release coating layer has a weight increment range of 2%-30%, preferably 4%-15%, more preferably 5%-10%.

6. The sustained-release pharmaceutical composition of topiramate according to claim 2, wherein the sustained-release coating layer comprises a sustained-release coating material, which is one or more selected from a group consisting of ethyl cellulose, Eudragit NE 30D, Eudragit RS 30D, Eudragit RL30D, preferably ethyl cellulose and/or Eudragit NE 30D, more preferably ethyl cellulose.

7. The sustained-release pharmaceutical composition of topiramate according to claim 2 or 6, wherein the sustained-release coating layer comprises ethyl cellulose and PVPK30.

8. The sustained-release pharmaceutical composition of topiramate according to claim 7, wherein the usage amount ratio of ethyl cellulose to PVP K30 is 1:0.20-1:0.45, preferably 1:0.25-1:0.40, particularly preferably 1:0.3-1:0.35.

9. A method for preparing the sustained-release pharmaceutical composition of topiramate of any one of claims 1-8, comprising the following steps:
a) providing an active drug of drug layer to perform drug-loading and coating a blank pellet core to obtain a drug-loading pellet;
b) subjecting the drug-loading pellet to sustained-release coating;
preferably, comprising the following steps:
a) providing topiramate and other adjuvants of drug layer, adding with a suitable amount of solvent for dissolution, and performing drug-loading and coating a blank pellet core to obtain a drug-loading pellet;
b) subjecting the drug-loading pellet to sustained-release coating;
more preferably, comprising the following steps:
a) providing topiramate and other adjuvants of drug layer, adding with a suitable amount of solvent for dissolution, and performing drug-loading and coating a blank pellet core with the drug solution, to obtain a drug-loading pellet;
b) dissolving a sustained-release coating material and other adjuvants of sustained-release coating layer in a solvent, subjecting the drug-loading pellet to sustained-release coating;
further preferably, comprising the following steps:
a) providing topiramate and other adjuvants of drug layer, adding with a suitable amount of solvent, heating and dissolving under stirring, providing a blank pellet core and placing in a fluidized bed coating pan for one-step granulation, performing drug-loading and coating with the above drug solution under stirring, to obtain a drug-loading pellet;
b) dissolving a sustained-release coating material and other adjuvants of sustained-release coating layer in a solvent, heating and dissolving under stirring, mixing homogeneously, passing through a 100 mesh sieve, to obtain a sustained-release coating solution;
c) taking the drug-loading pellet, spraying the sustained-release coating solution on surface of the drug-loading pellet in a fluidized bed, to obtain a sustained-release pellet of topiramate.

10. A use of the sustained-release pharmaceutical composition of topiramate according to any one of claims 1-8 in manufacture of a medicament for prophylaxis and/or treatment and/or adjunctive treatment of migraine, epilepsy, diabetes, dysneuria, depression, psychosis, headache, or hypertension.

11. A method for prophylaxis and/or treatment and/or adjunctive treatment of migraine, epilepsy, diabetes, dysneuria, depression, psychosis, headache, or hypertension, comprising a step of administering an effective amount of the sustained-release pharmaceutical composition of topiramate according to any one of claims 1-8.
